(19) United States
(12) Patent Application Publication
(10) Pub. No.: US 2020/0054467 A1
(43) Pub. Date: Feb. 20, 2020

(54) METHOD FOR ESTABLISHING AN ELECTRICALLY CONDUCTIVE ARTIFICIAL NERVE

(71) Applicant: Zakary Llamo-Cohen, Bryan, TX (US)

(72) Inventor: Zakary Llamo-Cohen, Bryan, TX (US)

(21) Appl. No.: 16/105,940

(22) Filed: Aug. 20, 2018

(51) Int. Cl. A61F 2/72; A61N 1/05; A61L 27/38

(52) U.S. Cl. CPC A61F 2/72; A61L 27/383; A61N 1/0551

(57) ABSTRACT

A method is provided for forming an electrically conductive pathway between the nervous system of a patient and a prosthetic device. The method includes implanting an electrically conductive scaffolding around a nerve in the body of the patient; attaching the scaffolding to an electrical conduit which electrically transmits the action potential of the nerve; inducing growth of the nerve about the scaffolding and the electrical conduit, thereby forming an electrically conductive pathway; and bringing the electrically conductive pathway into electrical communication with the prosthetic device.

### FIELD OF THE DISCLOSURE

The present disclosure relates generally to the treatment of nerve damage and to prosthetic devices, and more particularly to methods for establishing electrically conductive pathways between the injured segments of a patient's nerve, or between the nervous system of a patient and a prosthetic device.

### BACKGROUND OF THE DISCLOSURE

Nerve guidance conduits (also referred to as artificial nerve conduits or artificial nerve grafts) are an artificial means for guiding axonal regrowth in order to facilitate nerve regeneration by connecting the distal and proximal sections of an injured nerve, and are one of several clinical treatments that have been developed in the art for treating nerve injuries. In many cases, direct suturing of the two stumps of a severed nerve cannot be accomplished without tension. In such cases, the standard clinical treatment for peripheral nerve injuries is autologous nerve grafting, or using the patient's own nerve tissue for the treatment. More recently, advances in cellular processing has opened the opportunity for using donor tissue to bridge the gap through an allograft. Due to the limited availability of donor tissue and functional recovery in autologous nerve grafting, neural tissue engineering research has focused on the development of bioartificial nerve guidance conduits as an alternative treatment, especially for large defects.

The creation of nerve guidance conduits is frequently referred to as entubulation, since it involves enclosing the nerve ends and an intervening gap within a tube comprising biological or synthetic materials. This tube may be a biological tube, a synthetic tube or a tissue-engineered conduit. It is designed to facilitate neurotropic and neurotrophic communication between the proximal and distal ends of the nerve gap, to block external inhibitory factors, and to provide a physical guidance for axonal regrowth. The most basic objective of a nerve guidance conduit is to combine physical, chemical, and biological cues under conditions that will foster tissue formation.

Various materials have been utilized as nerve guidance conduits. Biologic tubes have included blood vessels and skeletal muscles. Non-absorbable synthetic tubes have been made from silicone, and bin-absorbable synthetic tubes have been made from polyglycolide. Tissue-engineered nerve guidance conduits have been fabricated from various scaffold structured, scaffold materials, cellular therapeutic agents, neurotrophic factors and biomimetic materials. The choice of which physical, chemical and biological cues to use is often based on the properties of the nerve environment, which is critical in creating the most desirable environment for axon regeneration. Factors commonly considered in material selection include biocompatibility, biodegradability, mechanical integrity, controllability during nerve growth, implantation and sterilization ability.

### BRIEF DESCRIPTION OF THE DRAWINGS

FIG. 1 is an illustration of a particular, non-limiting embodiment of a nerve tether in accordance with the teachings herein, and showing the tether at initial implantation.

FIG. 2 is an illustration showing the nerve tether of FIG. 1 after healing.

FIG. 3 is a side view illustration of a base plate for the nerve tether of FIG. 1.

FIG. 4 is a front view illustration of a base plate for the nerve tether of FIG. 1.

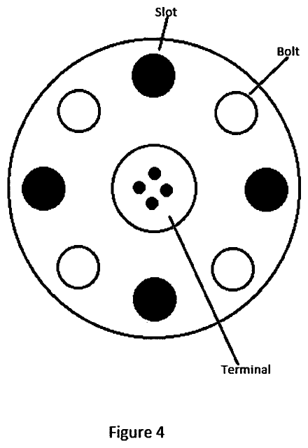

### SUMMARY OF THE DISCLOSURE

In one aspect, a method is provided for forming an electrically conductive pathway between the nervous system of a patient and a prosthetic device, or as a treatment for traumatic injuries to the nervous system. The method comprises (a) implanting an electrically conductive scaffolding around a nerve in the body of the patient; (b) attaching the scaffolding to an electrical conduit which electrically transmits the action potential of the nerve; (c) inducing growth of the nerve about the scaffolding and the electrical conduit, thereby forming an electrically conductive pathway; and (d) bringing the electrically conductive pathway into electrical communication with the prosthetic device or distal section of nerve.

### DETAILED DESCRIPTION

Nerve guidance conduits (NGCs) are artificial conduits for directing nerve growth, and are the first treatment option for nerve injuries that cannot be sutured without tension because they create a span the gap between the injured ends of the nerve, allowing the tissue to regrow. These NGCs are made up of a three-level scaffold: the superstructure, microstructure, and nanostructure. The superstructure is responsible for shaping the NGC and directing tissue growth. There are a variety of techniques used in generating the superstructure, but they generally consist of a combination of hydrogel, synthetic polymer, biological polymer, and/or Glial cells due to these material's low-impact on the body and regenerative properties. The microstructure and nanostructure are responsible for further influencing the cellular response to the scaffold and can affect multiple processes, including differentiation, adhesion, orientation, and expression.

On its own, a regenerated nerve will regrow at a rate of approximately 1mm per day, if there is an available pathway between the proximal and distal nerve stumps. With a sufficiently large gap, Wallerian Degeneration can lead to scarring and loss of tissue that can be irrecoverable during the time it takes for the gap to close. Even if the nerve recovers, the brain will need time to relearn how to use the affected area, and if immobility due to the injury leads to joint stiffness, the affected limb will be permanently weakened.

The development of an artificial nerve graft that will be able to conduct action potentials on its own instead of relying on full nerve regrowth to reestablish the pathway between the proximal and distal stumps will help to mediate the issues listed above by blocking degeneration and preventing extended loss of communication between the brain and the affected limb.

The use of electronics has become very common in human prosthesis as they allow more precise controls and increased power output of traditional mechanical prosthesis. Myoelectric limbs have been developed which control the limbs by converting muscle movements to electrical signals. In such devices, myoelectric signals are picked up by electrodes implanted in the muscle or wrapped around the nerve, and the detected signal is integrated. Once the integrated signal exceeds a certain threshold, the prosthetic limb control signal is triggered by the prosthetic's CPU and converted into commands for the robotic limb.

While the use of myoelectric controls is a notable advance in the art, such controls suffer from an inherent lag, and thus do not offer the type of direct force feedback experienced with a natural limb. These devices are also limited due to their complexity; they are especially susceptible to the environment dust, water, etc. and their power requirements place limits on the length of use. There is thus a need in the art for prosthetic devices which offer the advantages of myoelectric controls, without the inherent lag.

It has now been found that the foregoing needs may be addressed with the systems, methodologies and devices disclosed herein. In a preferred embodiment, these systems, methodologies and devices utilize a nerve tether to form a direct conduit between a nerve in the body of a patient and the electronic controls of a prosthetic device. Prosthetic devices may be made with this approach that do not suffer from the lag inherent in the myoelectric prosthetic devices developed to date, and that offer a feel and responsiveness which more closely resembles that experienced with a human limb.

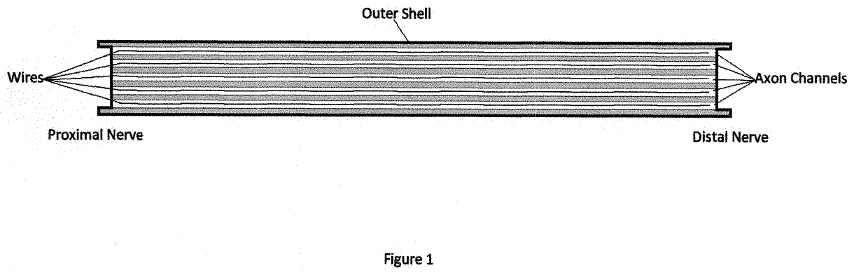

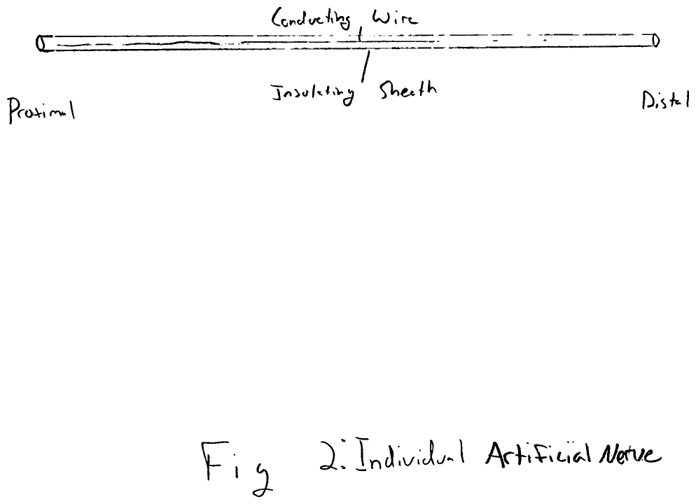

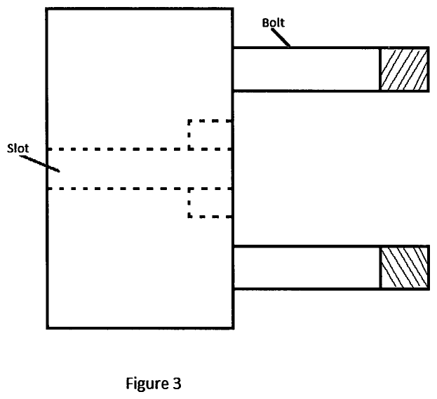

FIGS. 1-3 depict a first particular, non-limiting, embodiment of a nerve tether in accordance with the teachings herein. The nerve tether may be implanted between the distal and proximal ends of severed nerve, and by conducting action potentials and stimulating nerve regrowth, can be used as a bridge to prevent loss of function to the extremity.

The nerve tether of FIG. 1 may also form an electrically conductive pathway between the nervous system of a patient and the electronic components of a prosthetic device which has been fitted to the patient

The nerve tether of FIG. 1 may be formed by the steps of implanting an electrically conductive scaffolding around a nerve in the body of the patient, attaching the scaffolding to an electrical conduit which electrically transmits the action potential of the nerve, inducing growth of the nerve about the scaffolding and the electrical conduit, thereby forming an electrically conductive pathway, and bringing the electrically conductive pathway into electrical communication with the prosthetic device.

Various scaffolding materials may be utilized in the systems, devices and methodologies disclosed herein. These include, but are not limited to, materials selected from the group consisting of porous silicon, silicone, poly(3,4-ethylenedioxythiophene) (PEDOT) nanofibers, poly(3,4-ethylenedioxythiophene) polystyrene sulfonate (PEDOT:PSS) nanofibers, carbon nanotubes, polyaniline nanofibers, poly(D,L-lactic-co-glycolic acid) (PLGA), poly(L-lactic acid) (PLLA), and poly(c-caprolactone) (PCL). Preferably, the scaffolding material has a composition that promotes nerve growth.

Various conduit materials may be utilized in the systems, devices and methodologies disclosed herein. These include, but are not limited to, materials selected from the group consisting of porous silicon, silicone, PEDOT nanofibers, PEDOT:PSS nanofibers, carbon nanotubes, polyaniline nanofibers, PLGA, PLLA, and PCL.

Various means may be utilized in the systems, devices and methodologies disclosed herein to induce the growth of the nerve about the scaffolding or the electrical conduit. These include, but are not limited to, the application of glial cells or neurotropic factors to the scaffolding or electrical conduit. Preferably, inducing growth of the nerve about the scaffolding or the electrical conduit includes directing the nerve growth along a predetermined pathway.

Various configurations may be utilized in the systems, devices, and methodologies disclosed herein to create the form of the scaffolding or electrical conduit. These include, but are not limited to, the of use longitudinally oriented channels to create separate pathways for each axon in the nerve and insulate the wire from one another. Preferably, the scaffold will then incorporate a combination of cellular therapies, through the use of Glial cells, and neurotrophic factors to promote nerve growth.

The above description of the present invention is illustrative, and is not intended to be limiting. It will thus be appreciated that various additions, substitutions and modifications may be made to the above described embodiments without departing from the scope of the present invention. Accordingly, the scope of the present invention should be construed in reference to the appended claims.

What is claimed is:

A1. A method for forming an electrically conductive pathway between the distal and proximal ends of a damaged nerve in the patient's body, the method comprising:
implanting an electrically conductive scaffolding around the proximal end of a damaged nerve in the body of the patient;
attaching the scaffolding to an electrical conduit which electrically transmits the action potential of the nerve;
inducing growth of the nerve about the scaffolding and the electrical conduit, thereby forming an electrically conductive pathway; and
bringing the electrically conductive pathway into electrical communication with the distal end of said damaged nerve.

A2. The method of claim A1, wherein the conduit is used to recreate the naturally occurring neural pathway to function as a surgical treatment for moderate to extreme cases of nerve damage.

A3. The method of claim A1, wherein the scaffolding comprises a material selected from the group consisting of porous silicon, silicone, poly(3,4-ethylenedioxythiophene) (PEDOT) nanofibers, poly(3,4-ethylenedioxythiophene) polystyrene sulfonate (PEDOT:PSS) nanofibers, carbon nanotubes, polyaniline nanofibers, poly(D,L-lactic-co-glycolic acid) (PLGA), poly(L-lactic acid) (PLLA), and poly(ε-caprolactone) (PCL).

A4. The method of claim A1, wherein the electrical conduit comprises a material selected from the group consisting of porous silicon, silicone, PEDOT nanofibers, PEDOT:PSS nanofibers, carbon nanotubes, polyaniline nanofibers, PLGA, PLLA, and PCL.

A5. The method of claim A1, wherein the composition of the scaffolding promotes nerve growth.

A6. The method of claim A1, wherein inducing growth of the nerve about the scaffolding and the electrical conduit includes applying glial cells to the scaffolding and electrical conduit.

A7. The method of claim A1, wherein inducing growth of the nerve about the scaffolding and the electrical conduit includes applying neurotropic factors to the scaffolding and electrical conduit.

A8. The method of claim A1, wherein inducing growth of the nerve about the scaffolding and the electrical conduit includes directing the nerve growth along a predetermined pathway.

B1. A method for forming an electrically conductive pathway between the nervous system of a patient and a prosthetic device, the method comprising:
implanting an electrically conductive scaffolding around a nerve in the body of the patient;
attaching the scaffolding to an electrical conduit which electrically transmits the action potential of the nerve;
inducing growth of the nerve about the scaffolding and the electrical conduit, thereby forming an electrically conductive pathway; and
bringing the electrically conductive pathway into electrical communication with the prosthetic device.

B2. The method of claim B1, wherein the prosthetic device is equipped with control circuitry, and wherein bringing the electrically conductive pathway into electrical communication with the prosthetic device includes bringing the electrically conductive pathway into electrical communication with the control circuitry.

B3. The method of claim B1, wherein the prosthetic device is constructed using actuators or other such movement controller that can be triggered directly by the action potential transmitted along the tether.
